(19) United States
(12) Patent Application Publication
(10) Pub. No.: US 2009/0177048 A1
(43) Pub. Date: Jul. 9, 2009

(54) MINIATURE, WIRELESS APPARATUS FOR PROCESSING PHYSIOLOGICAL SIGNALS AND USE THEREOF

(75) Inventors: Terry B.J. Kuo, Taipei (TW); Cheryl C.H. Yang, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL YANG-MING UNIVERSITY, Taipei City (TW)

(21) Appl. No.: 12/404,774

(22) Filed: Mar. 16, 2009

Related U.S. Application Data
(63) Continuation of application No. 11/755,964, filed on May 31, 2007, now abandoned.

(51) Int. Cl. A61B 5/00

(52) U.S. Cl. 600/300

(57) ABSTRACT

The present invention provides an apparatus and method for processing plural of physiological signal input from external sensors are received by the signal-receiving element in a synchronized pattern.

### CROSS-REFERENCE TO RELATED APPLICATIONS

This application is a Continuation-In-Part of U.S. patent application Ser. No. 11/755,964 filed on May 31, 2007, now pending, and is hereby incorporated by reference in its entirety. Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of all of the prior arts of record and any search that the Office deems appropriate.

### FIELD OF THE INVENTION

The present invention relates to a miniature, wireless apparatus for processing physiological signals and use thereof.

### DESCRIPTION OF PRIOR ART

Physiological signals such as ECG, EEG, breath, and body temperature are signs of health. If combine and analyze the above physiological signals as well as the electromyogram, one can obtain the indications of sleep and autonomic nervous system. Collection and analysis of these physiological signals facilitates the understanding and medical application of numerous medical information. Particularly, the design of wireless remote measurement can reflect the physiological phenomena with high accuracy and low interference and provide the important information for precise understanding of various physiological functions.

Sleep medicine has breakthrough in the past five years. Some chronic sleep-related diseases including obstructive sleep apnea are getting more and more attention. Many medical researches also indicate that sleep troubles are probably one of the factors of hypertension. The number of sleep-related research and clinical examination has remarkably increased in recent years. It is thus evident that the sleep-related research is one of the emphases in future medical developments. However, the slow progress of sleep research at present medical environment made the sleep a key leak in the clinical healthcare. So far, the deficiency and poor establishment of the long-term monitoring instruments and analyzing tools resulted in the low willingness of patients as well as the limitation of the sleep-related medical development.

The key defects of existing sleep-medicine detecting system are described as follows:
(1) the constraint of traditional wired system:

The existing long-term physiological detecting system was mainly established on the basis of wired transmission technologies. Patients should paste a lot of electrodes on his or her body parts, and then the electrodes are connected to the signal amplifier via conducting wire for digital-to-analog conversion and digital signal processing. It was very awkward to operate the traditional wired system. The movement of patients was highly constrained due to the wiring all over the body. Even going to the toilet was inconvenient under the examination. As a result of all above disamenities, many patients hesitate to go for examination or refuse to cooperate with doctors for long-term inspection.
(2) the high price and difficulty of operation of traditional instruments:

Due to the need of professional technicians for operating long-term physiological signal detection system, the efficiency of sleep examination in hospitals is low. After a period of training the sleep examination could be administered smoothly.
(3) Recently, a few wireless instruments are under development, but the convenience of operation need to be improved.

Some manufacturers have launched so-called wireless system on the market, but most of the wireless systems are still limited by the tiresome conducting wires. The electrodes should be connected to the host by wire. After amplification and digital-to-analog conversion, the digital signals are then emitted by microcontroller and radio module. The whole system must be connected by conducting wires. Although the above system contributes to some improvement and convenience for the patients, those wires to some extent still restrict the movement of patients. Moreover, the wires themselves are the origin of various noise signals, causing the reduced accuracy of the examination result. In addition, the existing systems are unable to receive and process different physiological signals in one channel simultaneously, resulting in the squander of bandwidth and electricity. The fact that bulky instruments are not easy to carry is still another problem.

To sum up, the existing sleep detection systems have some inherent disadvantages. The conducting wires not only lead to the inconvenience of patients but inevitably increase the origins of noise signal. Additionally, these instruments are expensive, difficult to operation, and too bulky to monitor the patients' condition momentarily. Hence, developing a totally wireless, inexpensive, and handy physiological signal recording and examination system for personal operation and long-term monitoring is necessary and urgent. Then a physiological signal examination system with more convenience and less noise signals can be realized for monitoring patients' condition, evaluating the effect of operation, and further understanding the course of disease.

Patent No. 090128786 of R.O.C provided a sensor for simultaneously detecting electrocardiogram signal, pulsation, and acoustic wave from the neck. Patent application No. 091106492 of R.O.C provided a non-invasive autonomic nervous monitoring system and use thereof. Patent application No. 092117250 of R.O.C. provided an electrocardiogram signal converter and analog-to-digital converting device thereof. U.S. Pat. No. 6,360,117 provided an electrocardiogram signal collecting apparatus.

### SUMMARY OF THE INVENTION

The present invention provides an apparatus for processing physiological signal comprising: (a) a signal-receiving element, and (b) a signal-processing element; wherein said signal-receiving element receives plural of signals input from external sensors and transmits the signals to said signal-processing element; and said signal-processing element divides the receiving-time into n equal intervals and corresponds each divided time-interval to a signal detected by one sensor; and wherein said signal-receiving element sends a feedback signal on both a downlink to said external sensors and on an uplink from the external sensor so that said plural of signals input from external sensors are received by said signal-receiving element in a synchronized pattern.

The present invention further provides a method for processing physiological signals comprises receiving the signals by signal-receiving element, and dividing the receiving-time into n equal intervals by signal-processing element and corresponding each divided time-interval to a signal which is detected by one sensor, and in which the signal-receiving element sends a feedback signal on a downlink to the external sensors and on an uplink from the external sensors so that the plural of signals input from external sensors are received by the signal-receiving element in a synchronized pattern.

### BRIEF DESCRIPTION OF THE DRAWINGS

FIG. 1 indicates the apparatus for processing physiological signals combined with the signal sensor and the signal recorder.

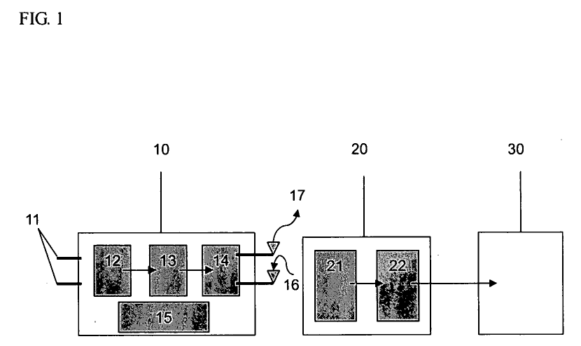

FIG. 2 indicates that multiple signal sensors transmits the detected signals synchronously to the apparatus for processing physiological signals.

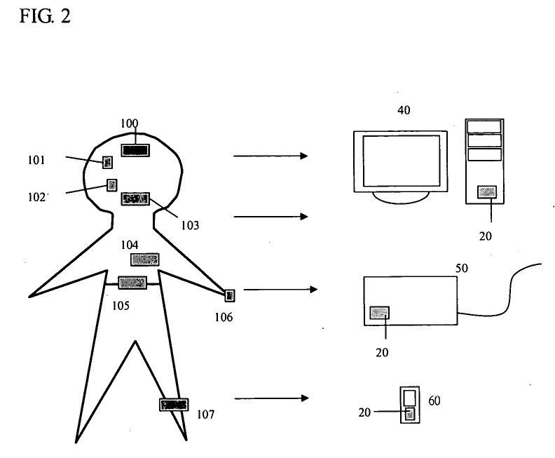

FIG. 3 indicates that the apparatus for processing physiological signals is integrated on a signal sensor.

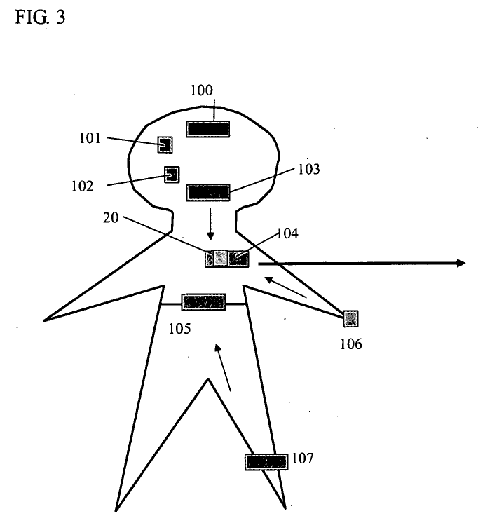

FIG. 4 indicates that the signal-processing element divides the receiving-time into 8 equal intervals and corresponds each divided time-interval to different signals detected.

FIG. 5 shows the result of the synchronous uplink and downlink signal transmission which proceed with 4 sensors.

### DESCRIPTION OF MAJOR PARTS IN THE PRESENT INVENTION

10: signal sensor
11: electrode
12: amplifier
13: microcontroller
14: transceiver module
15: power supply
16: uplink signal transmission
17: downlink signal transmission
20: signal processing apparatus
21: signal-receiving element
22: signal-processing element
30: signal recorder
100: wireless electroencephalogram sensor
101: wireless electrooculogram sensor
102: wireless electromyogram sensor
103: wireless temperature sensor
104: wireless electrocardiogram sensor
105: miniature tension sensor
106: blood oxygen saturation sensor
107: miniature acceleration sensor
40: personal computer
50: Internet
60: GSM mobile phone

### DETAILED DESCRIPTION OF THE INVENTION

### Term Definition

### Electroencephalogram (EEG)

A chart of the brain waves picked up by the electrodes placed on the scalp. Changes in brain wave activity can be an indication of REM sleep, consciousness, and nervous system disorders.

### Electrooculogram (EOG)

EOG is a technique for measuring the resting potential of the retina. The resulting signal is called the electrooculogram. The main applications are in opthalmological diagnosis and in recording eye movements. Unlike the electroretinogram, the EOG does not represent the response to individual visual stimuli.

### Electromyogram (EMG)

An electromyogram (EMG) is a test that is used to record the electrical activity of muscles. When muscles are active, they produce an electrical current. This current is usually proportional to the level of the muscle activity. An EMG is also referred to as a myogram.

### Electrocardiogram (ECG or EKG)

The electrocardiogram (ECG or EKG) is a noninvasive test that is used to reflect underlying heart conditions by measuring the electrical activity of the heart. By positioning leads (electrical sensing devices) on the body in standardized locations, information about many heart conditions can be learned by looking for characteristic patterns on the ECG.

### Firmware

In computing, firmware is software that is embedded in a hardware device. It is often provided on flash memory or as a binary image file that can be uploaded onto existing hardware by a user.

The present invention provides an apparatus for processing physiological signal comprising:

(a) a signal-receiving element, and
(b) a signal-processing element;
wherein said signal-receiving element receives plural of signals input from external sensors and transmits the signals to said signal-processing element; and said signal-processing element divides the receiving-time into n equal intervals and corresponds each divided time-interval to a signal detected by one sensor; and,
wherein said signal-receiving element sends a feedback signal on both a downlink to said external sensors and on an uplink from the external sensor so that said plural of signals input from external sensors are received by said signal-receiving element in a synchronized pattern.

The preferred embodiment of the present invention is signals from several external sensors can synchronous transmit uplink to the base and downlink to the external sensors.

In the present invention, n is ranged from 1 to 50. In the preferred embodiment, n is ranged from 1 to 30. In the more preferred embodiment, n is ranged from 1 to 20. In the best embodiment, n is ranged from 1 to 10.

The present invention further comprises one or more signal sensors which consist of electrode pair, amplifier, microcontroller, transceiver module, and power supply.

The electrode pair of the signal sensor is differential and comprises a positive electrode and a negative electrode which are connected to a person under test to collect a pair of physiological signals.

The amplifier of the signal sensor comprises a pair of input filters, a differential amplifier, and an output filter. The physiological signals collected from the positive electrode and the negative electrode have noise filtered out by the input filters to increase the signal-to-noise ratio, and then the physiological signals are differentially amplified by the differential amplifier. The differential amplifier attenuates the common mode noise of the pair of physiological signals, and simultaneously amplifies the differential part of the pair of physiological signals with appropriate magnification, so as to match the voltage range of the analog-to-digital conversion of the microcontroller.

The output filter filters out an amplified physiological signal that is over the Nyquist frequency (i.e., twice the sampling frequency of the analog-to-digital conversion of the microcontroller). Moreover, the impedance of the input end of the amplifier is larger than 200 kΩ, so as to prevent the leakage current caused by an operational error. The input filter and the output filter can be implemented by passive elements such as resistors or capacitors. The differential amplifier can be implemented by an operational amplifier or an instrumentation amplifier of the integrated circuit.

The microcontroller of the signal sensor comprises an analog-to-digital conversion unit and a digital signal processing unit. The analog-to-digital conversion unit performs an analog-to-digital conversion for the amplified physiological signal generated from the amplifier with the appropriate voltage resolution and sampling frequency, and then the digital signal processing unit performs a data compression for a digital physiological signal generated by the analog-to-digital conversion unit.

The transceiver module comprises a wireless transceiver and a modulator/demodulator. The input end of the transceiver module, being connected to the microcontroller, is a serial or parallel digital channel for receiving a digital physiological signal generated from the microcontroller. Then the modulator modulates the digital physiological signal compressed by the digital signal processing unit to a modulated physiological signal with the carrier frequency of 2.4 GHz. The modulated physiological signal is sent to a far end by the wireless transceiver in the form of a wireless physiological signal. Meanwhile, the wireless transceiver also receives a wireless signal from the far end, and then the wireless signal is demodulated by the demodulator to a digital data signal, and the digital data signal is transmitted to the microcontroller through the digital channel. The wireless signal sent from the far end comprises a control signal of the signal sensors and an acknowledgement signal sent by a signal-receiving element of the far end. The transceiver module performs wireless transmission and reception using the international industry, science, and medical (ISM) exclusive frequency band.

The signal receiving element of the apparatus provided in the present invention will guide the signal to the corresponding time-interval if the transceiver module doesn't send signal at corresponding time-interval. This guiding function makes it possible to corresponding detected signals from different sensors to the corresponding time-interval accurately. Therefore, a highly precise signal recording can be realized.

In the present invention, the radio interface is used as the transceiver module of signal sensor. The radio interface not only converts the digital physiological signals into radio signals as well as sending them but receives the radio signals inputted from outside.

In the present invention, the physiological signals include physiological signals transmitted by wired or wireless tools. Moreover, the physiological signals are human physiological signals including electrocardio-signal, electroencephalo-signal, electromyo-signal, electrooculo-signal, body temperature, tensile signal, and accelerative signal.

The signal sensor provided by the present invention is selected form the group consisting of wired/wireless electrocardiogram sensor, wired/wireless electroencephalogram sensor, wired/wireless temperature sensor, wired/wireless electromyogram sensor, miniature wired/wireless tension sensor, and wired/wireless acceleration sensor.

There are many open circuits of above sensors for free use and reference, including the collection of electro-signals, body temperature, blood oxygen concentration, and the circumference of thorax.

The apparatus of the present invention further comprises a remote signal recorder. In a preferred embodiment, the remote signal recorder is hard disc, floppy disc, miniature hard disc, or flash memory card which records and saves a large number of various processed physiological signals via wireless transmission for further analysis.

The apparatus of the present invention is carried out by micro-computer system such as personal computer, notebook computer, radio station, or personal digital assistant, which can further analyze the collected data or deliver the collected data to other signal-receiving elements. Additionally, the results of analysis can be transmitted to other micro-computer systems via the internet. The apparatus provided by the present invention also collects, saves, and transmits the sleeping data as a peripheral device of a portable computer system. Data analysis in the present invention is carried out by sleep-analyzing algorithm and autonomic nervous-analyzing algorithm.

The signal-receiving element of the apparatus can further send a feedback signal to transceiver module in all of signal sensors, making the transceiver module send signals in a synchronized pattern. The synchronization favors the subsequent comparison and calculation in signal analysis and therefore a more accurate evaluation and diagnosis can be obtained.

In the present invention, the physiological signal processing apparatus can be further integrated into the signal sensors. By the concept of System-on-a-Chip (SoC), various functions including signal detection, reception, and processing can be totally integrated on a single chip set. Consequently, the trend of miniaturization and multi-function in electronic instruments can be realized. The radio signals emitted by other signal sensors can be simultaneously collected, combined, and saved in the remote signal recorder by the integrated system. An entirely wearable physiological signal monitoring system can be obtained. All the wireless physiological signal sensors and the signal recorder are put on the patient so that one can go around at will. Moreover, all kinds of physiological signals such as EEG and ECG under sleeping can be analyzed with minimum disturbance to the activities of daily livings. The system can be used to monitor the course of chronic diseases and to evaluate the effects of operation, the sleeping quality, and the autonomic nervous function.

A method for processing physiological signals comprises receiving the signals by signal-receiving element, and dividing the receiving-time into n equal intervals by signal-processing element and corresponding each divided time-interval to a signal which is detected by one sensor, and in which the signal-receiving element sends a feedback signal on a downlink to the external sensors and on an uplink from the external sensors so that the plural of signals input from external sensors are received by the signal-receiving element in a synchronized pattern.

In the present invention, n is ranged from 1 to 50. In the preferred embodiment, n is ranged from 1 to 30. In the more preferred embodiment, n is ranged from 1 to 20. In the best embodiment, n is ranged from 1 to 10.

The method provided by the present invention can be used to synchronously collect the signals emitted by several signal sensors in a single frequency channel. In addition to the efficient use of limited bandwidth, the method combined with the existent digital wireless transmission technologies would reaches the optimized application. Due to the simplified structures, the synchronous data-collection can be carried out in a more power-saving condition.

The method provided by the present invention further comprises a signal-detecting step by signal sensors and a signal-recording step by a signal recorder. The signal-receiving element can further send a feedback signal to the transceiver module in all of the signal sensors, making transceiver module send signals in a synchronized pattern.

The method provided by the present invention can be combined with wireless physiological detection technology, synchronous emission/receiving technology, synchronous recording/saving technology, and sleep-analyzing algorithm in order to fulfill a totally wireless and simple-to-use physiological signal monitoring system for accurate and real time analysis. The monitoring system can be used in evaluation of sleep quality, diagnosis of sleep obstacles, assessment of effect of hypnotics, evaluation of side effect to sleep and autonomic nervous function caused by various drugs, assessment of influences on sleep and autonomic nervous function due to various regimen and health-improving methods, evaluation of influences on sleep and autonomic nervous function caused by taking health food, and assessment of sleeping condition of elders and new-born infants.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

### EXAMPLES

The following examples are offered by way of illustration and not by way of limitation.

### Example 1

### Synchronous Signal Collection by Multiple Wireless Sensors

Various signal sensors 10 were pasted on the particular body parts for collecting different physiological signals. The wireless electroencephalogram sensor 100 was put on forehead for collecting EEG. The wireless electrooculogram sensor 101 was put on the canthus for collecting EOG The wireless electromyogram sensor 102 was put on the corners of the mouth or chin for collecting EMG. The wireless temperature sensor 103 was put beneath the nostrils for collecting the temperature of breath as an indication of snort. The wireless electrocardiogram sensor 104 was put on the chest for collecting ECG. The miniature tension sensor 105 was put on the pectoral for evaluating the respiratory function. The blood oxygen saturation sensor 106 was put on the finger for detecting the blood oxygen saturation. The miniature acceleration sensor 107 was put on the body or legs for collecting accelerative signals as a quantitative indication of movement or postures.

### Example 2

### Signal Receiving of Multiple Wireless Sensors

In order to obtain a high-quality result of physiological signal analysis, the signals of multiple signal sensors 10 should be emitted and received in a synchronous manner. In the present invention, the firmware was embedded on the microcontroller 13 of every signal sensor 10. Not only the transceiver module 14 was involved in the present invention but the signal receiving element 21 received all the emitting signals and carried out the synchronous control. Furthermore, flash memory was built in the signal sensor 10 of the present invention for extended storage of the collected physiological signal and increased flexibility.

### Example 3

### Signal Recording of Multiple Wireless Sensors

The signals collected by the signal sensors 10 were transmitted to signal-receiving element 21 by wireless transmission, or recorded in the remote signal recorder 30 of signal sensors 10. In spite of the convenience brought by the wireless transmission, the wireless signal transmission was prohibited in many places such as hospitals. Interferences in the environment may also reduce the quality of wireless signal transmission. The signal recorder 30 of the present invention contained a Non-Volatile Random Access Memory for recording various physiological signals. Even if the power failure or poor signal transmission occur, the signals recorded by the signal recorder 30 will not be affected. The flexibility of use and the possibility of long-term signal collection of were further increased by the continuity of the recording function provided by the physiological signal monitoring system.

### Example 4

### The Principle of Signal Processing of Multiple Wireless Sensors

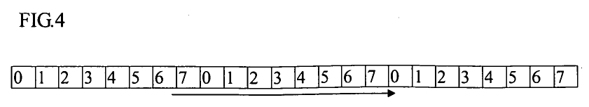

The signal-processing element 22 in the signal processing apparatus 20 divided the receiving-time into n equal intervals and assigned numbers to the intervals. In the best embodiment, there are eight different signal sensors 10 so the signal-processing element 22 divided the receiving-time into eight equal time intervals and each of the intervals was assigned from 0 to 7 (FIG. 4). Each time interval could only receive signals emitted from one particular signal sensor 10. For instance, time interval 0 could only receive the signals emitted from the signal sensor 100; time interval 1 could only receive the signals emitted from the signal sensor 101; time interval 2 could only receive the signals emitted from the signal sensor 102, and so forth.

If any one of the signal sensors 10 failed to send signals at corresponding time-interval, the signal receiving element 21 will guide the signal to the corresponding time-interval. In addition, the signal-receiving element 21 could further send a feedback signal to the transceiver module 14 in all of the signal sensors 10, making the transceiver module 14 send signals in a synchronized pattern. The whole course of synchronization was conducted by the signal receiving element 21, and all of the signal sensors 10 were also finely tuned by the signal receiving element 21. Accordingly, the signals could be still kept in a perfectly synchronized pattern even after a long-term recording.

### Example 5

### Protocol for Practicing Synchronous Operation of Uplink and Downlink Signal Transmission in 4 Miniature Wireless Sensors

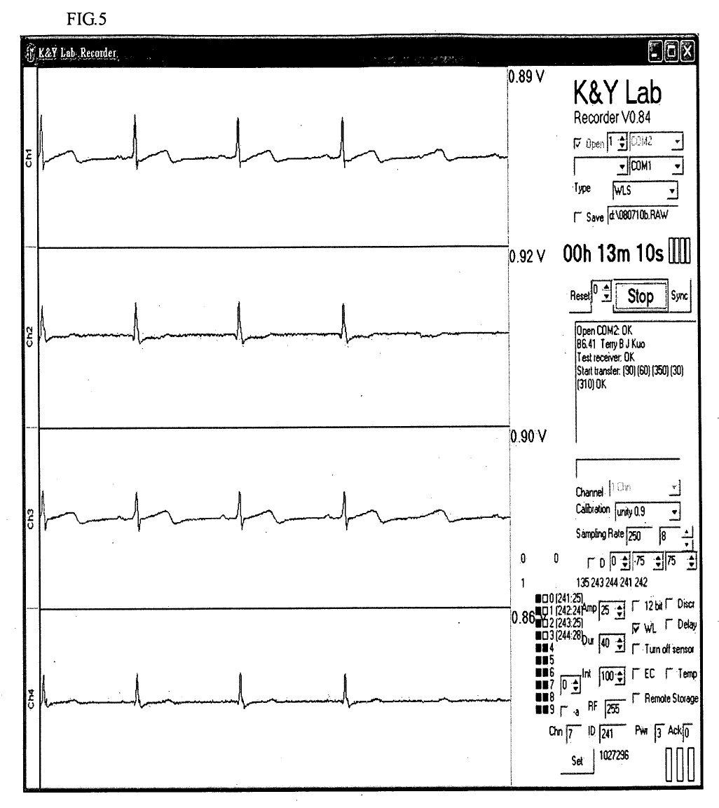

As shown in FIG. 5, the result by the following steps stated the synchronous uplink and downlink signal transmission which proceeded with 4 external sensors.

### Steps:

1. Synchronizing all external sensors and the base, and each sensor had its time slot (e.g. sensor 1 had time slot 1, sensor 2 had time slot 2, sensor 3 had time slot 3 . . . , the base had time slot 0).
2. All external sensors listened to the base in time slot 0.
3. The base transmitted a ‘START’ command to all external sensors in time slot 0.
4. All external sensors started to synchronize when received the ‘START’ command from the base.

What is claimed is:

1. An apparatus for processing physiological signal comprising:
(a) a signal-receiving element, and
(b) a signal-processing element;
wherein said signal-receiving element receives plural of signals input from external sensors and transmits the signals to said signal-processing element;
and said signal-processing element divides the receiving-time into n equal intervals and corresponds each divided time-interval to a signal detected by one sensor; and,
wherein said signal-receiving element sends a feedback signal on both a downlink to said external sensors and on an uplink from the external sensor so that said plural of signals input from external sensors are received by said signal-receiving element in a synchronized pattern.

2. The apparatus as claimed in claim 1, wherein said n is ranged from 1 to 50.

3. The apparatus as claimed in claim 1, which further comprises one or more signal sensors.

4. The apparatus as claimed in claim 3, wherein said signal sensor is selected form the group consisting of wireless electrocardiogram sensor, wireless electroencephalogram sensor, wireless temperature sensor, wireless electromyogram sensor, miniature tension sensor, and acceleration sensor.

5. The apparatus as claimed in claim 3, wherein said signal sensor consists of electrode, amplifier, microcontroller, transceiver module, and power supply.

6. The apparatus as claimed in claim 5, wherein said signal receiving element guides the signal to the corresponding time-interval if said transceiver module does not send signal at corresponding time-interval.

7. The apparatus as claimed in claim 5, wherein said transceiver module is radio interface.

8. The apparatus as claimed in claim 3, which further comprises a signal recorder.

9. The apparatus as claimed in claim 8, wherein said signal recorder is hard disc, floppy disc, miniature hard disc, or flash memory card.

10. The apparatus as claimed in claim 1, which is carried out by a micro-computer system including at least one member selected from the group consisting of personal computer, notebook computer, radio station, and personal digital assistant.

11. The apparatus as claimed in claim 1, which further analyze the collected data or deliver the collected data to other signal-receiving elements.

12. The apparatus as claimed in claim 11, wherein the data analysis is carried out by sleep-analyzing algorithm and autonomic nervous-analyzing algorithm.

13. The apparatus as claimed in claim 3, wherein said signal sensors is integrated by system-on-a-chip.

14. The apparatus as claimed in claim 1, wherein said physiological signals includes physiological signals transmitted by wired or wireless tools.

15. The apparatus as claimed in claim 1, wherein said physiological signal is human physiological signal.

16. A method for processing physiological signals comprising:
(a) receiving the signals by signal-receiving element; and
(b) dividing the receiving-time into n equal intervals by signal-processing element and corresponding each divided time-interval to a signal which is detected by one sensor; and,
wherein said signal-receiving element sends a feedback signal on a downlink to said external sensors and on an uplink from said external sensors so that said plural of signals input from external sensors are received by said signal-receiving element in a synchronized pattern.

17. The method as claimed in claim 16, which further comprises a signal-detecting step by signal sensors.

18. The method as claimed in claim 16, which further comprises a signal-recording step by a signal recorder.

19. The method as claimed in claim 16, which is used in evaluation of sleep quality, diagnosis of sleep obstacles, assessment of effect of hypnotics, evaluation of side effect to sleep and autonomic nervous function caused by various drugs, assessment of influences on sleep and autonomic nervous function due to various regimen and health-improving methods, evaluation of influences on sleep and autonomic nervous function caused by taking health food, and assessment of sleeping condition of elders and new-born infants.
